(19) United States
(12) Patent Application Publication
(10) Pub. No.: US 2006/0074413 A1
(43) Pub. Date: Apr. 6, 2006

(54) Method and apparatus for substantial and uniform ablation about a linear bipolar array of electrodes

(76) Inventor: Kamran Behzadian, Sunnyvale, CA (US)

Correspondence Address: INHOUSE IP, 280 COLORADO AVE, PALO ALTO, CA, 94301, US

(21) Appl. No.: 11/286,832

(22) Filed: Nov. 23, 2005

Related U.S. Application Data
(63) Continuation-in-part of application No. 10/878,168, filed on Jun. 28, 2004.

(51) Int. Cl. A61B 18/14

(52) U.S. Cl. 606/41

(57) ABSTRACT

The described invention relates to a medical device for substantially sized uniform ablation of animal or human tissue comprising of a bipolar generator for generating radio frequency at an electrode, a polarity alternator, a probe having a handle, and an elongated member, and a tip at the far end of the distal end suitable for insertion into tissue. The elongated member has a proximal and distal end with an electrode cluster of three or more electrodes located on the distal end of the elongated member. The electrodes are electrically insulated from each other and at least two of the electrodes have dissimilar polarity from each other wherein at least one of the electrodes has a high voltage polarity and at least one of the electrodes has a return polarity. During ablation, the polarity alternator changes the polarity of the electrodes to cycle lesion formation along the length of the electrode cluster repeatedly from electrode to electrode to form a spherical or near spherical lesion at the distal tip equal in diameter to the length of the cluster.

### CROSS-REFERENCE

This application is a continuation-in-part of currently pending U.S. patent application Ser. No. 10/878,168, filed on Jun. 25, 2004, which is fully incorporated by reference herein.

### FIELD

The invention relates generally to a method and apparatus for utilizing energy, such as radio frequency energy, in a multi electrode and bipolar fashion to treat defined substantial volumes of animal or human tissue uniformly about a linear array of electrodes and more particularly have the ability to concentrate lesion formation around desired electrodes through the use of a member having multiple electrodes whose polarity of one or more electrodes is independently controlled and dynamically changed along the length of the electrodes and repeated as many times as necessary to produce a substantial and uniform lesion.

### BACKGROUND

Methods for treating damaged animal or human tissue, such as those with benign and malignant tumors, have been developed and improved for many years. Recently, a new technique known as radio frequency ablation has been developed in order to treat damaged tissue by destroying its damaged cells plus the adjacent undamaged cells to prevent further spreading. The radio frequency energy causes the tissue to heat up to a high temperature, therefore breaking apart and killing the cells. The objective of radio frequency ablation is to heat tissues to 50-100 degrees centigrade for 4-6 minutes without causing charring or vaporization. Under these conditions there is almost instantaneous cellular protein denaturation, melting of lipid bilayers and destruction of DNA, RNA and key cellular enzymes. This is commonly known as cell necrosis.

In a monopolar ablation system, a probe or catheter containing electrodes with high voltage polarity that releases electrical energy, is placed inside the body while an electrode pad with return polarity that completes the electrical circuit is placed outside on the patient's skin. The result is greater amounts of electrical energy being dispersed throughout the patient's body, therefore causing collateral damage, or destroying tissue that is not targeted.

Following the development of this device is a new system known as a bipolar ablation system. This improved device contains both the electrodes with high voltage and return polarity on the same probe that is placed inside the tissue, therefore eliminating the need for an external return electrode pad and eliminate the possibility of collateral damage. However, bipolar instruments available in the market are limited in lesion size and uniformity.

Monopolar radiofrequency ablation instruments for substantial ablation marketed by Tyco, Boston Scientific and others produce spherical or near spherical lesions of 2 to 5 cm in diameter, delivered around a single needle shaft of 1.25 to 2.5 mm diameter with over 100 watts of electrical energy. These monopolar instruments require dispersion of electric energy throughout the body and cause collateral damage such as burns around the return electrode pads, commonly known as pad-burns.

In bipolar ablation systems, the electric energy is present local to the ablated area and not dispersed throughout the body, but their use is still limited due to limitations in lesion geometry and size.

U.S. Pat. Nos. 6,312,428, 6,524,308 and 6,706,039 describe devices for electro thermal cauterization of tissue with linear electrodes. When operated in a bipolar mode, these devices are limited to spherical lesions of 2 cm or smaller. See FIG. 5. As the inter electrode spacing is increased or as pairs of electrodes are added in series in an attempt to increase the lesion size, the lesion becomes elliptical or becomes two lobes. Therefore, cauterization zones, or electro thermal lesions made per the arts are limited in size to under 2 cm when spherical or near spherical in geometry and not comparable to monopolar devices in the market.

U.S. Pat. No. 6,447,506 describes a device for creating long, thin lesions. When operated in a bipolar mode, this device produces long and thin lesions that are about twice as wide as the body width and as long as the electrode cluster. Typical embodiments produce a lesion of 5 cm by 2.5-3 mm wide.

While technology has resulted in our ability to ablate targeted volumes of tissue, there is still an important need for a bipolar ablation system that heats a zone about a linear cluster of electrodes on a single shaft uniformly, and produces a substantially sized uniform lesion that is nearly spherical and similar in size and uniformity to lesions produced by currently available single needle monopolar radiofrequency ablation instruments.

An improved bipolar single needle device for substantial uniform ablation would have to produce uniform lesions that are nearly spherical in geometry, with a diameter of 2 to 5 cm around a single shaft similar to monopolar radiofrequency ablation systems.

The present invention provides such methods that will in turn enable practitioners to ablate a significantly greater amount of targeted areas of tissue uniformly about a single needle bipolar linear array of electrodes combined with avoiding the unnecessary destruction of other healthy tissue.

### SUMMARY

The invention described is a medical device for substantial and uniform ablation of animal or human tissue comprising of a generator for generating radio frequency electric energy at an electrode, a polarity alternator for dynamically and automatically altering the polarity of individual electrodes in a linear cluster of electrodes, a probe having a handle, a tip tapered to provide a sharp point, and an elongated member. The elongated member has a proximal end and a distal end. At the distal end, there is a linear electrode cluster of three or more electrodes wherein the electrodes are electrically insulated from each other and at least two of the electrodes have dissimilar polarity from each other. At least one of the electrodes has a high voltage polarity and at least one of the electrodes has a return polarity.

During ablation, polarity alternator is used for dynamically and automatically changing the polarity of the electrodes in a linear cluster individually at the tip at the far end of the distal end that is suitable for insertion into tissue. The electrodes are optionally spaced evenly or optionally unevenly along the length of the distal end of the elongated member.

The electrodes are optionally composed of electrically conductive matter including copper, stainless steel, or precious metal plated material.

The described invention consists of a method for substantially uniform ablation of animal or human tissue further comprising the steps of placement of electrode cluster of three or more electrodes on the distal end of an elongated single member adjacent to the tissue. The electrodes are capable of carrying an electrical charge.

Optionally, the method activates a generator with high voltage and ground return polarities. Optionally, a polarity alternator that is located inside the generator, inside the probe handle, or a stand-alone instrument is activated to create dissimilar polarities between the three or more electrodes such that the total surface area of the electrodes with a high voltage polarity is unequal to the total surface area of the electrodes having a return polarity, creating a higher current density about the electrodes of polarity with a lesser total surface area sufficient to cause ablation of tissue adjacent to the area of higher current density.

Optionally, the polarity of the three or more electrodes is altered individually to create a higher current density about the electrodes or polarity having a lesser surface area at a different point along the length of the cluster of electrodes prior to altering the polarity of electrodes, to ablate tissue adjacent to the area of higher current density.

The alteration of the polarity of the three or more electrodes will optionally change individually from high voltage to return polarity, neutral, or remain at high voltage polarity and from return to high voltage polarity, neutral, or remain at return polarity.

Lastly, as an option, the polarity alteration of the three or more electrodes is repeated during ablation and lesion formation is cycled over the length of the cluster and from electrode to electrode as many times as needed to achieve a spherical and uniform lesion of the tissue that is similar in size and uniformity to lesions made by monopolar instruments.

REFERENES CITED [REFERENECED BY]

| | |
|---|---|
| 2003/0199862 | Simpson et al. |
| 2002/0193790 | Fleishman et al. |
| 2001/0008967 | Sherman et al. |
| 6,312,428 | Eggers et al. |
| 6,447,506 | Swanson et al. |
| 6,524,308 | Muller et al. |
| 6,706,039 | Muller et al. |

### BRIEF DESCRIPTION OF DRAWINGS

FIG. 1 shows a side block view of a bipolar radio frequency ablation system—Generator, Polarity Alternator and Probe.

FIG. 2A and 2B show a bipolar probe with an electrode cluster at the distal tip detailing one possible Polarity Alternator operation.

FIG. 3A, 3B, and 3C show side views of a three electrode clusters with individual electrodes energized with different polarities and resulting electric field and lesion concentration zones.

FIG. 4A and 4B show an embodiment with flexible coils with rectangular cross section coils with straight edges and a cut away view respectively.

FIG. 5 shows a small bipolar lesion.

FIG. 6 shows a substantial and uniform lesion.

FIG. 7 shows an embodiment of the device of the invention with 3 electrodes in the cluster of electrodes.

FIG. 8 shows uniform nature of lesion formation in a protein medium.

FIG. 9A and 9B show lesion formation in muscle and liver tissue respectively.

### DETAILED DESCRIPTION

This invention relates to a bipolar radio frequency ablation system and method. Radio frequency ablation may be performed through an open incision or through laparoscopy, which is performed through multiple small incisions, or percutaneously as required. The duration and power requirements of a radio frequency ablation procedure may depend on many factors, including the size of the needed lesion, number of desired applications and location of the animal or human tissue being treated.

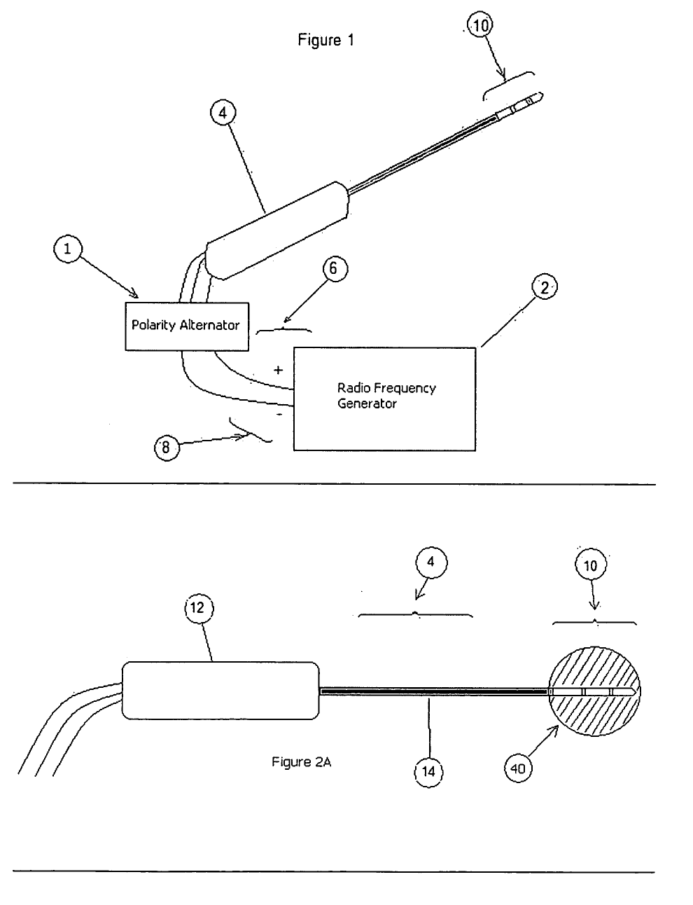

FIG. 1 shows a bipolar ablation system of the invention comprised of a bipolar generator 2, a polarity alternator 1, and a single needle bipolar probe 4 with an electrode cluster that consists of three or more electrodes at the distal tip 10. The bipolar generator 2 may be a conventional general purpose electrosurgical power supply operating at a frequency in the range from about 200 kHz to about 1.2 MHz, with a conventional sinusoidal or non-sinusoidal wave form. The bipolar generator 2 has a positively charged 6 high voltage polarity and a negatively charged 8 return polarity. Such power supplies are available from many commercial suppliers and control power output based on temperature, current, voltage, or impedance feedback from the probe or on an activation time basis. The polarity alternator 1 allocates electrical connection to individual electrodes in the cluster 10 and is capable of altering individual electrodes from high voltage polarity to return polarity or neutral and from return polarity to high voltage polarity or neutral and dynamically repeating the polarity alterations during ablation.

The probe 4, as shown in FIG. 2A is comprised of a handle 12, an elongated member 14, and a linear electrode cluster with three or more electrodes 10. Formation of a substantial uniform lesion 40 is shown around the linear cluster 10.

FIG. 2B shows high voltage and return polarities of a radiofrequency generator into the polarity alternator. Polarity alternator 1 changes polarity of individual input to a cluster of three electrodes as shown in table in FIG. 2B.

Polarity alternator 1 alters the individual polarities to the electrode array during ablation so that lesion is initially formed about electrode E3 then electrode E2 then electrode E1 for a preset time period T, and then lesion formation is repeated about electrodes E3, E2, E1 and then back to E3 so on. As such, lesion formation repeating about electrodes E3, E2, and E1 and then back to E3 is referred to as cycle of lesion formation.

FIG. 2B shows only one possible method of polarity alteration. In the example method shown, E2 always remains at high voltage polarity while polarity of other electrodes alters.

Polarity alternator 1 is shown as a stand-alone instrument in FIG. 2B, however it may optionally be located inside the generator 2 or inside handle 12.

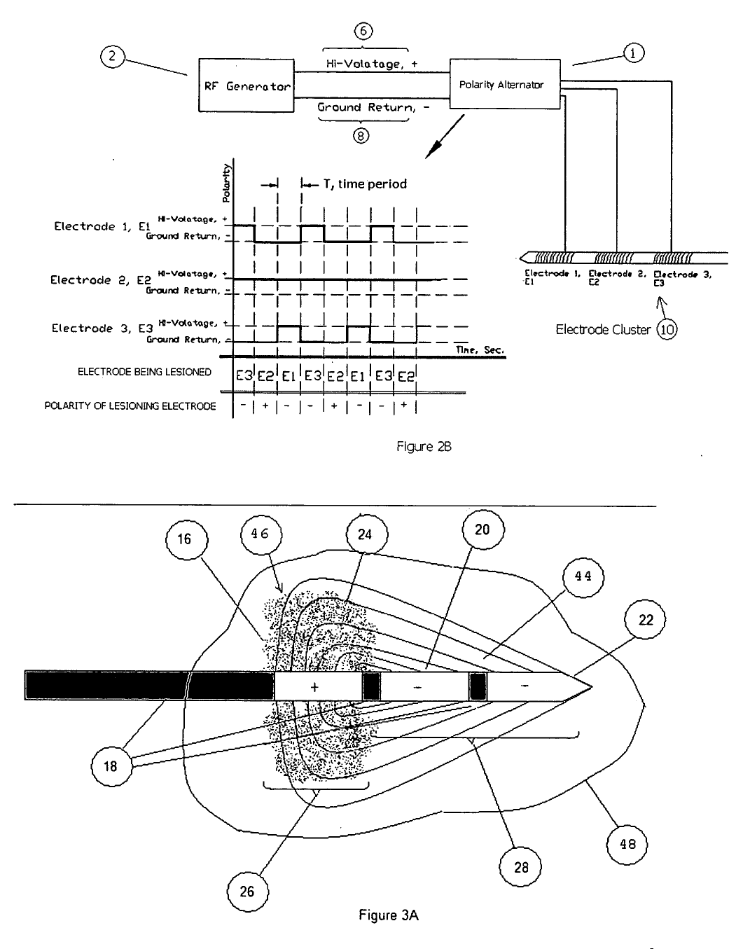

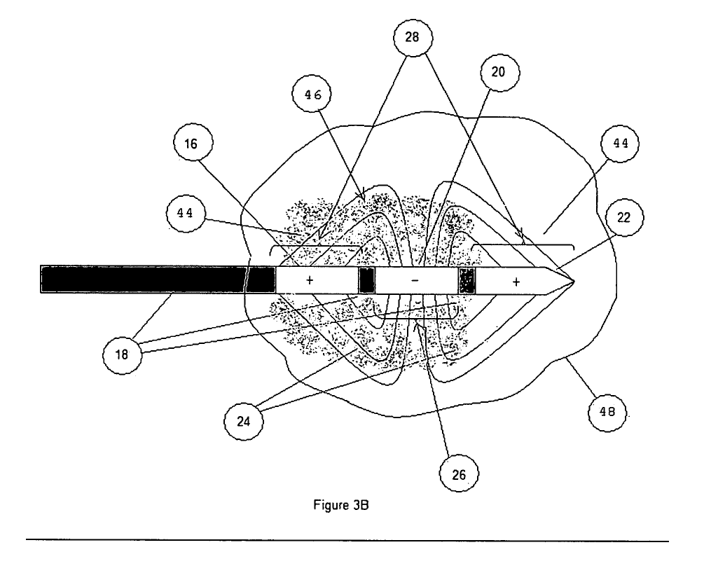

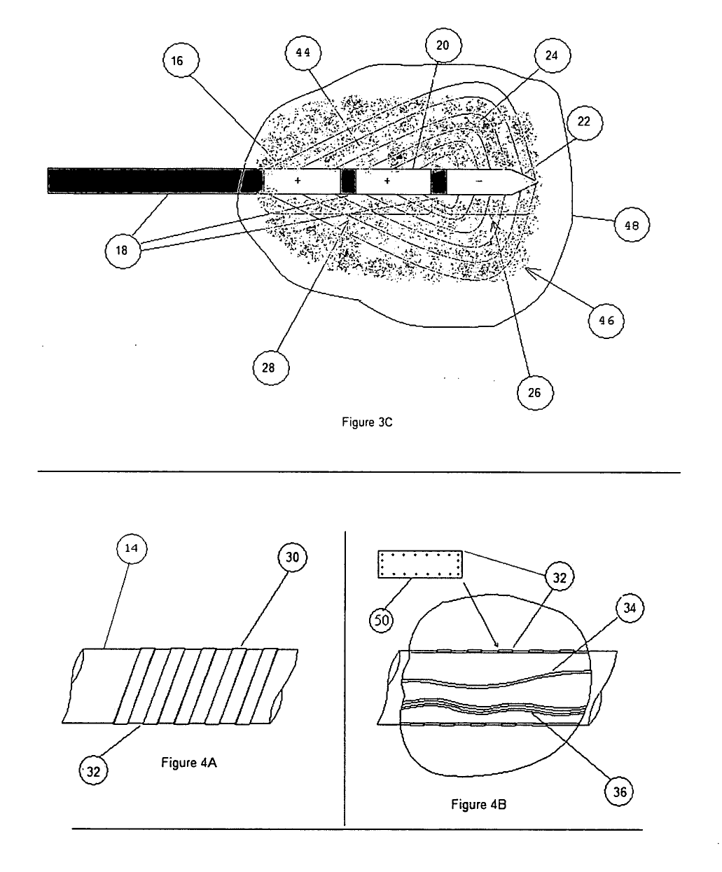

FIGS. 3A through 3C are examples of steps of ablating tissue by means of a bipolar probe 4 with an electrode cluster that consists of three or more electrodes at the distal tip 10 and with a resulting electric field when electrodes are identical in composition and geometry. Alteration of polarity is repeated to cycle lesion formation about individual electrodes during ablation and will ultimately result in a substantially sized spherical lesion.

FIG. 3A shows dissimilar polarity by electrode 16 being energized with high voltage polarity 6, which is denoted by a “+” sign, and electrodes 20 and 22 being energized with return polarity 8, which is denoted by a “−” sign. Item 18 are electrical insulation and can be found between the electrodes and on the proximal end of the probe. Dissimilar surface areas, shown with smaller surface area 26 and larger surface area 28, allows the electric field and therefore electrical current density to be higher 24 about electrode 16 and lower 44 about electrodes 20 and 22, therefore producing a higher current density 24 and lower current density 44, thus ablation heat generation is greatest about electrode 16 resulting in lesion generation 46 in targeted tissue 48 about electrode 16 that has a high voltage polarity and for a time period, T. The electrodes may be spaced evenly or unevenly with respect to electrical insulation 18 found in between each electrode.

Next step is represented in FIG. 3B. FIG. 3B shows dissimilar polarity by electrode 20 being energized with return polarity, − and electrodes 16 and 22 being energized with high voltage polarity, +. Item 18 are electrical insulation. Dissimilar surface areas allow the electric field and therefore electric current density to be higher 24 about electrode 20 and lower 44 about electrodes 16 and 22, therefore producing a higher current density 24 and lower current density 44, thus ablation heat generation is greatest about electrode 20 resulting in lesion generation 46 in targeted tissue 48 about electrode 20 which has a return polarity, − and for a time period T. The electrodes may be spaced evenly or unevenly with respect to electrical insulation 18 found in between each electrode.

Next step is represented in FIG. 3C. FIG. 3C shows dissimilar polarity by electrode 22 being energized with return polarity and electrodes 16 and 20 being energized with high voltage polarity. Item 18 are electrical insulation. Dissimilar surface areas allow the electric field and therefore electric current density to be higher 24 about electrode 22 and lower 44 about electrodes 16 and 20, therefore producing a higher current density 24 and lower current density 44, thus ablation heat generation is greatest about electrode 22 resulting in lesion generation 46 in targeted tissue 48 about electrode 22 which is has a return polarity, − for a time period T. The electrodes may be spaced evenly or unevenly with respect to electrical insulation 18 found in between each electrode.

Lesion formation is independent of polarity because it forms where a higher current density is present. Additionally, current density is independent of polarity and a function of active surface area. Lesion will form about the electrode or electrodes with the higher current density around them and irrespective of a particular polarity. Therefore, electrodes with high voltage polarity or return polarity in FIGS. 3A through 3C may have their polarities altered in order to shift high current density to the tissue surrounding a different electrode and independent of electrode polarity.

The polarity alteration is dynamically repeated to cycle lesion formation through the three electrodes as shown in FIG. 3A, 3B and 3C during ablation per table shown in FIG. 2B. Lesion will initially form as a long and thin volume about the linear array of electrodes during the first few cycles of lesion formation and eventually becomes a near sphere upon repeated cycling of lesion formation about individual electrodes over the length of the array. The spherical or near spherical lesion that is formed has a diameter equal to the length of the electrode cluster and is uniform.

In one embodiment, a cluster 10 of three 12 mm long electrodes separated by 5 mm length of insulation each on a 1.75 mm shaft 14 with a lesion formation time period T of 5 to 10 seconds about each electrode in the cycle will produce a spherical or near spherical lesion of diameter 45 to 50 mm with 12 to 15 watts of input radiofrequency energy in about 15 minutes of ablation time.

In another embodiment, a cluster 10 of three 3 mm long electrodes separated by 1 mm length of insulation each on a 0.5 mm shaft 14 with a lesion formation time period T of 2 to 3 seconds about each electrode in the cycle will produce a spherical or near spherical lesion of diameter 10 to 12 mm with about 2 to 5 watts of input radiofrequency energy in about 4 minutes of ablation time.

In yet another embodiment, a cluster 10 of three 1 mm long electrodes separated by 1 mm length of insulation each on a 0.5 mm shaft 14 with a lesion formation time period T of 0.5 to 1.5 seconds about each electrode in the cycle will produce a spherical or near spherical lesion of diameter of 5 mm with about 1 to 2 watts of input radiofrequency energy in about 1 to 2 minutes of ablation time.

It is appreciated that polarity alteration shown in table in FIG. 2B is one possible method of dynamically repeating lesion formation about a linear array of three electrodes and cycling lesion formation over the length of the electrodes during ablation. It is appreciated that there are many formats for dynamically changing polarities in a linear bipolar array of electrodes to repeatedly cycle lesion formation over the length of an array of electrodes in order to produce a substantially sized uniform lesion of spherical or near spherical geometry.

FIG. 4A shows a single electrode made up of flat wire with sharp and straight edges 32 wrapped about the circumference of the elongated member 14 in the form of a flexible coil 30. A higher local current density 24 is present about the straight edges 32.

FIG. 4B shows a rectangular cross section 50 of the flexible coiled electrode 30 with straight edges 32 and temperature feedback thermocouple wires 34 for individual electrodes.

An electrode shaped in the form of flexible coils 30 with straight edges 32 wrapped about the circumference of the elongated member 14 with rectangular cross section 50 will produce more uniform ablations over the length of the electrode in addition to being flexible. Although flexible coils with non-rectangular cross sections such as round may be used to achieve flexibility, a rectangular shape is preferred because an individual electrode made up of many straight edges 32 spaced closely to each other produces a more uniform ablation that is made up of smaller lesions about the straight edges 32 that are spaced closely to each other that propagate and join into a uniform lesion about the entire length of the individual electrode. The power input to the electrode array is controlled by monitoring lesion temperature by locating feedback temperature sensors such as thermocouple wires 34 under electrodes. Power is adjusted to maintain an average temperature under 100 degrees centigrade around the linear array.

Determination of lesion formation and completion is accomplished by monitoring electrical current and voltage in individual wire connections 36 to electrodes in circuitry embedded within the generator 2 or polarity alternator 1. Electrical characteristics such as impedance of the tissue under treatment are calculated by the circuitry based on the monitored voltage and current. The circuit continuously monitors and calculates the electrical characteristics of the lesion that is being formed around the electrode where lesion is being formed and the other electrodes and then averages the values over a cycle for all electrodes in a cluster.

Lesion is formed as tissue cell necroses takes place. As cells change composition while the tissue is heated, the average impedance of the volume of tissue where electric current is contained continuously increases until full necroses of all cells in the volume of tissue where electric field is present takes place. Lesion grows to the size of the electric field because that is where electrons responsible for ablation are present. As lesion is formed and grows to the size of the electric field, the average impedance of tissue where electric energy is present increases until full lesion formation. Average impedance measured between electrodes does not increase any further when a lesion the size of the electric field about a linear array of electrodes is formed. Substantial and uniform ablations 40 only treat the material portions of tissue that is located in a sphere or near-sphere around the distal tip of the probe 4 and equal in diameter to the length of the bipolar cluster 10 as shown in FIG. 2A.

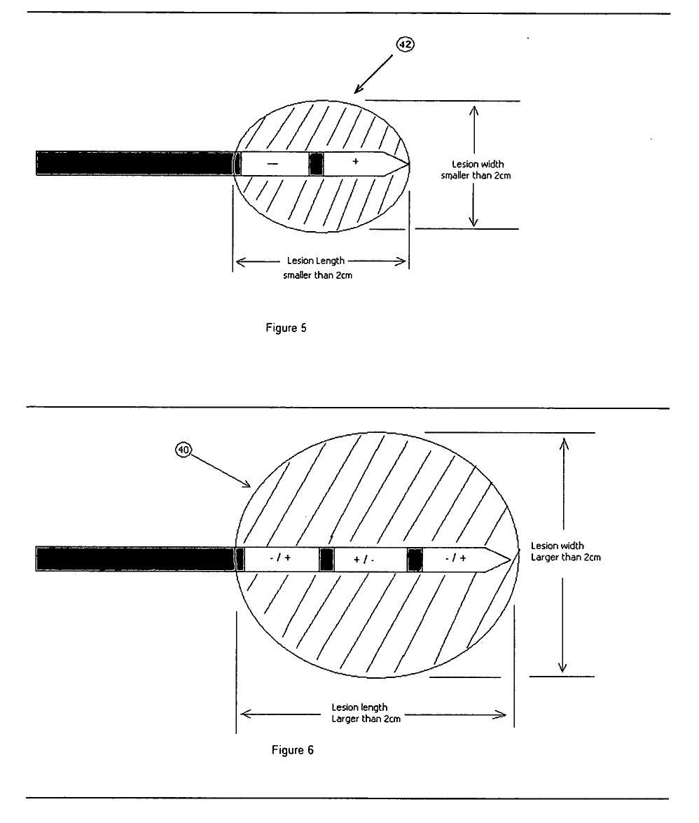

FIG. 5 shows a spherical or near spherical lesion made by prior art, U.S. Pat. Nos. 6,312,428, 6,524,308 and 6,706,039. Spherical or near spherical lesions made by prior art are under 2 cm in width or length. Addition of electrode pairs, increasing the length of the electrodes, or increasing the space between electrodes does not affect lesion width and may only increase lesion length.

FIG. 6 shows a uniformly heated zone around a bipolar array of electrodes made per disclosed art. Uniform and substantial lesions are spherical or near spherical with lesion widths equal or near-equal to lesion length and therefore spherical or near-spherical with diameters ranging from 5 mm to 10 cm.

The ability to control lesion formation about specific electrodes in a bipolar device and cylce lesion formation over the length of a cluster of electrodes repeatedly by alteration of polarities enables the device to produce a substantial uniform ablation lesion 40 by manipulating current densities about electrodes in a linear array of electrodes and cycling the current density and therefore lesion formation many times over the length of the cluster resulting in a substantial uniform ablation of the tissue along the length of the electrode cluster that is spherical or near spherical with a diameter about equal to the length of the electrode cluster. See FIG. 6.

### EXAMPLE

Lesion formation per this patent may be verified in many mediums such as a transparent protein medium such as egg white or in animal tissue such as muscle tissue, liver tissue, kindeny tissue, lung tissue, etc.

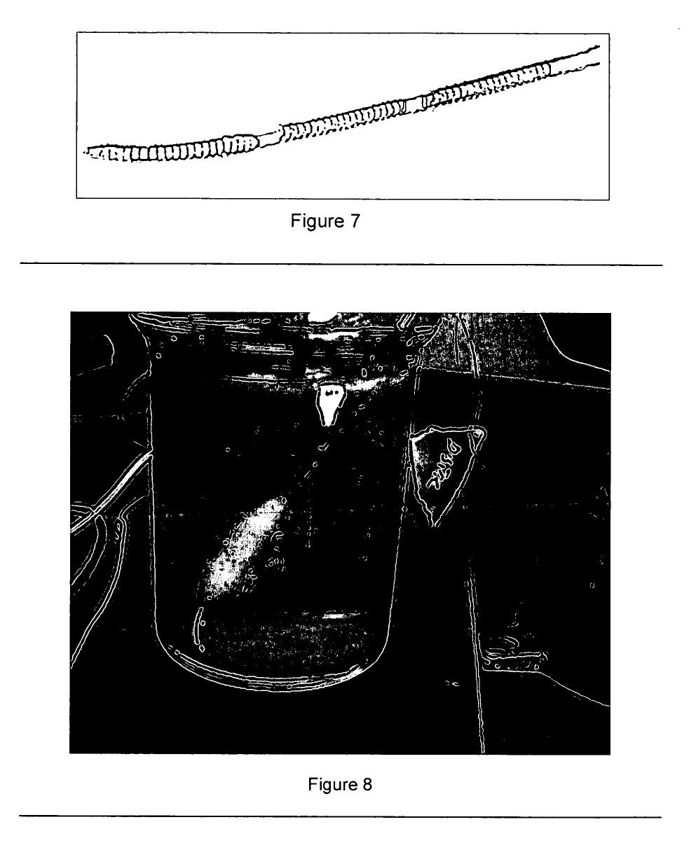

FIG. 7 shows a flexible embodiment of the device of the invention with 3 electrodes in the cluster of electrodes. Individual electrodes are about 2 cm long and are separated from each other by 5 mm. Each electorde is made up of wire with rectangular cross section that is wrapped around the body of the probe.

FIG. 8 shows the uniform nature of lesion formed over the length of an individual electrode made with rectangular cross section. Polarity of the three electrodes of the probe in FIG. 7 are arranged so lesion is formed about the middle electrode, E2.

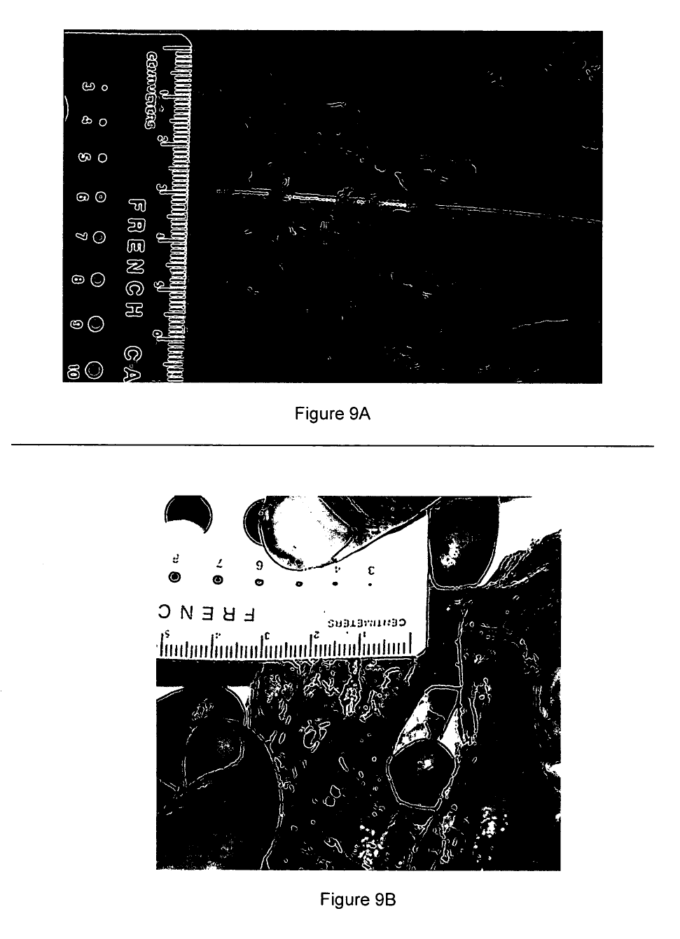

FIG. 9A shows a lesion in muscle tissue formed per this invention. Lesion is produced and then the tissue is sectioned in order to show the uniform and substantial nature of the lesion. Lesion is produced by cycling the lesion formation at T=3 sec per electrode, for a total time of approximately 15 minutes, or about 100 repeated cycles of lesion formation along the length of the electrode array. Lesion formation about each electrode is repeated many times in a cycled fashion from electrode to electrode along the length of the cluster per the polarity alteration method of FIG. 2B where a substantial uniform spherical lesion is produced by repeated cycling of lesion formation about E3, E2, E1, and then back to E3, and so on throughout ablation. Lesion is spherical and has a diameter of approximately 50 mm that is about equal to the length of the three-electrode cluster of 48 mm. In this example electrodes are 13 mm long and are separated by 5 mm of insulation on a shaft of 1.75 mm. Lesion is formed with about 15 watts of input radiofrequency energy.

A lesion in liver tissue is shown in FIG. 9B. Lesion is produced and then tissue is sectioned in order to show the uniform nature of the lesion. Lesion is made around a 20 mm long electrode cluster made up of three each 5 mm long electrodes separated by 2.5 mm length of insulation on a shaft of 1.5 mm with a lesion formation time period of T=2 seconds about each electrode in the cycle. Lesion is made in about 6 minutes of ablation time or 60 repeated cycles of lesion formation about the length of the electrode cluster. Resulting lesion is spherical with a diameter of about 20 mm and equal to the length of the electrode cluster, and it was formed with about 5 watts of radiofrequency energy.

From the aforementioned description, it is appreciated how the objectives and features of the above-described invention are met. The invention provides a linear cluster on a single shaft as a minimally invasive surgical tool and technique for heating substantial volumes of tissue about the electrode elements uniformly to produce a substantial and uniform lesion that is spherical or nearly spherical equal in diameter to the length of the linear electrode cluster located at the distal end of the apparatus of the invention. It is appreciated that various modifications of the apparatus and method are possible without departing from the invention, which is defined by the claims set forth below.

What is claimed is:

1. A medical device for substantially uniform ablation of animal or human tissue comprising:
a. a generator for generating radio frequency energy at an electrode;
b. a linear electrode cluster of at least three or more electrodes;
c. a polarity alternator for changing the polarity of the electrodes during ablation to form a lesion about an individual electrode;
d. said polarity alternator automatically changing the polarity of one or more electrodes to change lesion formation about a different electrode in the cluster;
e. said polarity alternator repeatedly cycling lesion formation over the length of the electrode cluster during ablation to create a substantial and uniform ablation about the cluster of electrodes.

2. The medical device of claim 1 further comprising a probe having a handle and an elongated member.

3. The medical device of claim 1 further comprising an elongated member having a proximal and a distal end.

4. The medical device of claim 1 where the electrodes are electrically insulated from each other.

5. The medical device of claim 1 where at least two of the electrodes have dissimilar polarity from each other.

6. The medical device of claim 1 where at least one of the electrodes having a high voltage and at least one of the electrodes having a return polarity.

7. The medical device of claim 1 wherein the time period to ablate the tissue to form a lesion about an individual electrode during the cycling of lesion formation is from 0.5 to 10 seconds.

8. The medical device of claim 1 wherein the time period to ablate the tissue to form a lesion about an individual electrode during the cycling of lesion formation is from 3 to 10 seconds wherein the ablation will form a spherical or near spherical lesion of diameter greater than 20 millimeters and up to 100 millimeters about the cluster of electrodes.

9. The medical device of claim 1 wherein the time period to ablate the tissue to form a lesion about an individual electrode during the cycling of lesion formation is from 0.5 to 3 seconds wherein the ablation will form a spherical or near spherical lesion of diameter less than 20 millimeters about the cluster of electrodes.

10. The medical device of claim 1 further comprising of a tip at the far end of the distal end suitable for insertion into tissue.

11. The medical device of claim 1 wherein the electrodes are spaced evenly along the length of the distal end of the elongated member.

12. The medical device of claim 1 wherein the electrodes are spaced unevenly along the length of the distal end of the elongated member.

13. The medical device of claim 1 wherein the elongated member is rigid or optionally flexible.

14. The medical device of claim 1 wherein the electrodes have dissimilar surface areas exposed to the tissue.

15. The medical device of claim 1 wherein the tip is tapered to provide a sharp point.

16. The medical device of claim 1 wherein the electrodes are composed of electrically conductive matter or precious metal-plated material.

17. The medical device of claim 1 wherein the electrodes are shaped in the form of a coil with rectangular cross section wrapped about the circumference of the elongated member.

18. The medical device of claim 17 wherein the form of a coil wrapped about the circumference of the elongated member allows flexibility of the electrode cluster.

19. The medical device of claim 1 wherein the lesion formation time period about each electrode is variable.

20. The medical device of claim 1 having a bend anywhere along the length of the electrode cluster.

21. The medical device of claim 1 wherein an elongated member has varying flexibility along the length.

22. A method for substantially uniform ablation of animal or human tissue further comprising the steps of:
a. placement of a linear electrode cluster of three or more electrodes adjacent to the tissue, the electrodes capable of carrying an electrical charge;
b. activating a generator to create dissimilar polarity between the three or more electrodes such that the total surface area of the electrodes with a high voltage polarity is unequal to the total surface area of the electrodes having a return polarity, creating a higher current density about the electrodes with a lesser total surface area sufficient to cause ablation of tissue adjacent to the area of higher current density;
c. automatically altering the polarity of the three or more electrodes individually to create a higher current density about the electrodes having a lesser surface area at a different point along the length of the cluster of electrodes prior to altering the polarity of electrodes, to ablate tissue adjacent to the area of higher current density to achieve uniform ablation of the tissue along the length of the cluster of electrodes;
d. repeatedly changing of polarity of the three or more electrodes individually to transfer lesion formation adjacent to a different electrode and repeating the polarity change to cycle lesion formation from electrode to electrode in a cluster of electrodes; and
e. changing the polarity of electrodes to cycle lesion formation over the length of the cluster or a section of the cluster repeatedly during ablation.

23. Method in claim 22 wherein the alteration of the polarity of the three or more electrodes will change individually from high voltage to return polarity, neutral, or remain at high voltage polarity and from return to high voltage polarity, neutral, or remain at return polarity.

24. Method of claim 22 wherein the time period to ablate the tissue adjacent to the area of higher current density during the cycling of lesion formation is from 3 to 10 seconds wherein the ablation will form a spherical lesion of diameter greater than 20 millimeters and up to 100 millimeters about the cluster of electrodes.

25. Method of claim 22 wherein the time period to ablate the tissue adjacent to the area of higher current density during the cycling of lesion formation is from 0.5 to 3 seconds wherein the ablation will form a spherical lesion of diameter less than 20 millimeters about the cluster of electrodes.
